(19) United States
(12) Patent Application Publication
(10) Pub. No.: US 2013/0066372 A1
(43) Pub. Date: Mar. 14, 2013

(54) SYSTEM CORRECTING SPINAL ORIENTATION THROUGH MUSCULAR BIO-ELECTRICAL SIGNAL ANALYSIS

(71) Applicant: Jong Kyu Yoon, Gwangyang (KR)

(72) Inventor: Jong Kyu Yoon, Gwangyang (KR)

(73) Assignee: RAPA & LIFE Co., Ltd., Wanju-gun (KR)

(21) Appl. No.: 13/255,607

(22) PCT Filed: Dec. 10, 2010
(86) PCT No.: PCT/KR2010/008825

§ 371 Date: Sep. 9, 2011

(30) Foreign Application Priority Data
Jun. 3, 2010 (KR) 10-2010-0052222

(51) Int. Cl. A61H 1/00; A61B 5/107; G06F 19/00; A61B 5/0488

(52) U.S. Cl. 606/237

(57) ABSTRACT

A system for correcting spinal orientation through an analysis of bio-electrical signals of muscles is provided. The system includes: an electromyography (EMG) signal processing module measuring bio-electrical signals of muscles related to a spinal disease and analyzing the same; an HCl control module generating a control signal for controlling a spinal orientation correction module based on information received from the EMG signal processing module and delivering the generated control signal to the spinal orientation correction module; the spinal orientation correction module correcting a mal-aligned spine of a patient to its correct orientation according to the control signal; and an information display and feedback module receiving information from the EMG signal processing module in real time, displaying the received information, and delivering information received from a user to the HCl control module.

### CROSS-REFERENCE TO RELATED APPLICATIONS

This application claims priority to International Application No. PCT/KR2010/008825, filed Dec. 10, 2010, and Korean Patent Application No. 2010-0052222, filed Jun. 3, 2010, each of which is incorporated by reference herein in its entirety.

### TECHNICAL FIELD

The present invention relates to a system for correcting spinal orientation through a muscular bio-electrical signal analysis and, more particularly, to a technique of measuring and analyzing muscular bio-electrical signal to recognize the condition and function of muscles and providing the results as quantitative numerical values to allow a healer (i.e., a physician, a therapist, or the like) to perform spinal orientation while monitoring the condition of the muscles of a patient undergoing spinal orientation treatment, to thereby decrease the possibility of a spinal mal-alignment such as intervertebral disk, scoliosis, and the like.

### BACKGROUND ART

A spinal disease, which has conventionally accounted for the largest portion of disorders of the musculoskeletal system, is very common in people spending long periods in a standing position, who are bound to experience at least a small amount of back pain, and these days, those who complain of a considerable disability in living daily life due to serious pain caused by a herniated intervertebral disk is increasing.

Such a spinal disease is caused as factors, such as a weight load, a ground reaction force (GRF), an arrangement of uncovertebral joints, muscular strength, muscle balance, and the like, are mal-aligned due to a bad habits or posture, exercise, or the like, causes the spine to be mal-aligned. There are various spinal diseases, including mal-alignment syndromes such as a herniated intervertebral disk, a degenerative disk, an abnormal spinal twist, temporomandibular joint dysfunction, scoliosis, muscular pain, and the like.

In order to alleviate diseases and pain due to mal-alignment syndromes and resolve spine joint dysfunction, namely, in order to make uncovertebral joints have a normal arrangement condition, the most important thing is treating the muscles for an arrangement of each on the spine, namely, skeletal muscles, such as muscles for maintaining a posture against gravity, muscles charged with the movement of spine, and the like.

### DISCLOSURE OF THE INVENTION

Conventionally, in order to treat skeletal muscles, a therapist performs manual therapy such as a chiropractic technique, or the like, based on his subjective medical determination, and merely provides a feedback method while monitoring only mechanical operation data by using a device performing traction, distraction, and decompression and a device implementing the range of motion (ROM) of spine.

An aspect of the present invention provides a system for correcting spinal orientation through an analysis of bio-electrical signals of muscles capable of measuring and analyzing bio-electrical signals of muscles to recognize the condition and function of muscles and providing the results as quantitative numerical value to allow a healer (i.e., a physician, a therapist, or the like) to perform spinal orientation while monitoring the condition of muscles of a patient in a spinal orientation treatment, to thereby increase a success rate of a spinal mal-alignment.

According to an aspect of the present invention, there is provided a system for correcting spinal orientation through an analysis of bio-electrical signals of muscles, including: an electromyography (EMG) signal processing module measuring bio-electrical signals of muscles related to a spinal disease and analyzing the same; an HCl control module generating a control signal for controlling a spinal orientation correction module based on information received from the EMG signal processing module and delivering the generated control signal to the spinal orientation correction module; the spinal orientation correction module correcting a mal-aligned spine of a patient to its correct orientation according to the control signal; and an information display and feedback module receiving information from the EMG signal processing module in real time, displaying the received information, and delivering information received from a user to the HCl control module.

According to another aspect of the present invention, there is provided a method for correcting a spinal orientation, including: measuring and analyzing bio-electrical signals of muscles related to a spine disease; generating a control signal for controlling a spinal orientation correction device according to the results obtained by analyzing the bio-electrical signal; and driving the spinal orientation correction device according to the control signal to correct a mal-aligned spine of a patient to its orientation.

As set forth above, according to exemplary embodiments of the invention, while a healer treats a spinal mal-alignment of a patient, he or she can monitor data obtained by analyzing electrical signals of the patient's muscles. Thus, the healer can feed back the analysis data to recognize the patient's condition and analyze the influence of a load applied by each function performed by a spinal orientation correction device on the patient's body to thus appropriately control the amount, duration, or the like, of the load.

Accordingly, the patient may feel comfortable while he is receiving the spinal correction treatment, and the patient's spinal mal-alignment can be precisely treated, increasing a success rate of the treatment.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 is a view showing a configuration of an electromyography (EMG) signal obtained by measuring bio-electricity generated according to a muscular activity;

FIG. 2 is a schematic block diagram of a system for correcting spinal orientation through an analysis of bio-electrical signals of muscles according to an exemplary embodiment of the present invention;

FIG. 3 is a detailed block diagram of a biometric information analyzing/processing unit illustrated in FIG. 2;

FIG. 4 is a graph for explaining an SEF analysis performed by a frequency domain analyzing unit illustrated in FIG. 3;

FIG. 5 is a graph for explaining an MEF analysis performed by the frequency domain analyzing unit illustrated in FIG. 3;

FIG. 6 is a graph for explaining a correlation analysis performed by the frequency domain analyzing unit illustrated in FIG. 3;

FIG. 7 is a graph for explaining an analysis process performed by a muscular stiffness analyzing unit illustrated in FIG. 3;

FIGS. 8 and 9 are graphs for explaining an analysis process performed by an MVIC normalizing/analyzing unit illustrated in FIG. 3; and

FIG. 10 is a flow chart illustrating a treatment process by the system for correcting a spinal orientation through an analysis of bio-electrical signals of muscles according to an exemplary embodiment of the present invention.

### BEST MODE FOR CARRYING OUT THE INVENTION

Exemplary embodiments of the present invention will now be described in detail with reference to the accompanying drawings. The invention may, however, be embodied in many different forms and should not be construed as being limited to the embodiments set forth herein. Rather, these embodiments are provided so that this disclosure will be thorough and complete, and will fully convey the scope of the invention to those skilled in the art. In the drawings, the shapes and dimensions may be exaggerated for clarity, and the same reference numerals will be used throughout to designate the same or like components.

It will be understood that when an element is referred to as being “connected with” another element, it can be directly connected with the other element or intervening elements may also be present. In contrast, when an element is referred to as being “directly connected with” another element, there are no intervening elements present. In addition, unless explicitly described to the contrary, the word “comprise” and variations such as “comprises” or “comprising”, will be understood to imply the inclusion of stated elements but not the exclusion of any other elements.

The term ‘module’ refers to a unit for performing a particular function or operation, which may be implemented by hardware, software, or the combination of hardware and software.

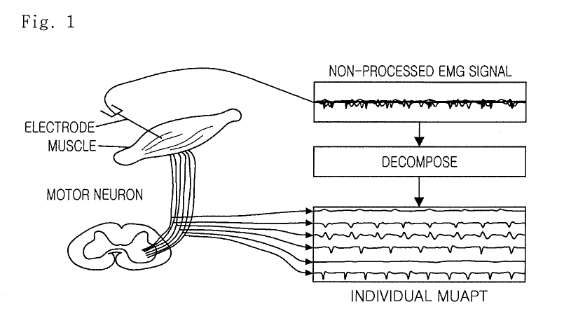

FIG. 1 is a view showing a configuration of an electromyography (EMG) signal obtained by measuring bio-electricity generated according to a muscular activity.

An EMG signal is obtained by measuring bio-electricity generated according to the activity of muscles covering a skeleton (or a frame) of a human's body that support and move the body, which is made up of motor unit action potentials (MUAPs) generated from a control signal of the cerebrum delivered to a muscular fiber, a basic component of muscle, through a nerve fiber.

The EMG signal includes information regarding a muscular fiber control mechanism of a nervous system according to the cerebrum, information regarding an action potential generation according to qualities of respective muscular fibers and transport properties, and the like. Thus, various types of information included in the EMG signal can be recognized by interpreting the EMG signal according to a proper method, whereby the condition of a neuro-muscular system can be diagnosed to be used as medical treatment information.

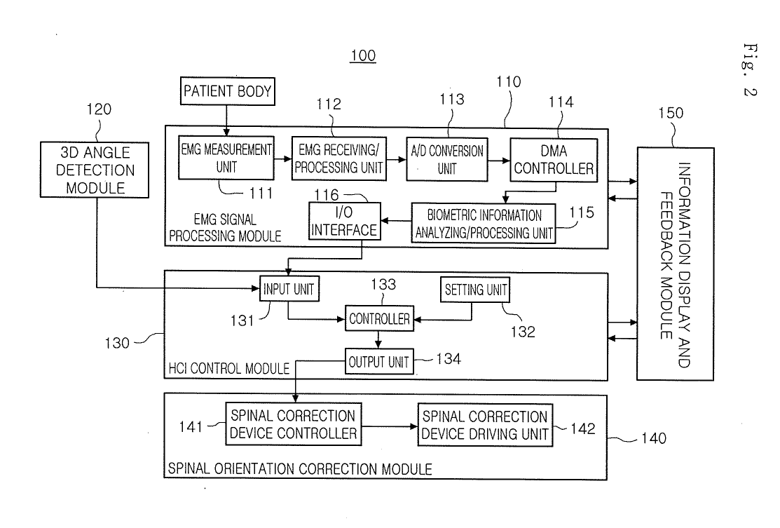

FIG. 2 is a schematic block diagram of a system for correcting a spinal orientation through an analysis of bio-electrical signals of muscles according to an exemplary embodiment of the present invention. The system for correcting a spinal orientation is configured to include an EMG signal processing module 110, a three-dimensional (3D) angle detection module 120, an HCl control module 130, a spinal orientation correction module 140, and an information display and feedback module 150.

The EMG signal processing module 110, which serves to measure and analyze bio-electrical signals of muscles related to a spinal disease of a patient, includes an EMG measurement unit 111, an EMG receiving/processing unit 112, an A/D conversion unit 113, a DMA (Digital Media Adapter) controller 114, a biometric information analyzing/processing unit 115, and an I/O interface 116.

The EMG measurement unit 111 detects bio-electrical signals of muscles by using a surface recording electrode, a static EMG scanner ceramic, or the like, on the relevant muscles to obtain information regarding a spinal disease.

The EMG receiving/processing unit 112, which serves to receive the signal detected by the EMG measurement unit 111 and preprocesses the signal, may include an amplifying unit for amplifying a fine signal received from the EMG measurement unit 111 to have a voltage level of, for example, 50 mV or higher and an analog filter unit for canceling noise. In detail, the received fine signal in unit of a micro-volt (uV) is amplified to unit of volts (mV) by a low-noise preamplifier and then sent to a main amplifier. The signal is amplified by the main amplifier to have a voltage level ranging from −2.5 V to 2.5 V. The amplified signal passes through an analog filter unit, whereby RF noise is canceled. In this case, a cutoff frequency of the analog filter unit may be regulated by a resister value and a capacitor value.

The A/D conversion unit 113 converts the analog signal, which has been preprocessed by the EMG receiving/processing unit 112, into a digital signal. In this case, preferably, the analog signal, which has been preprocessed by the EMG receiving/processing unit 112, is converted to have a level of 5 V by increasing a DC offset value by, for example, 2.5 V and then input to the A/D converter 113.

The DMA controller 114 transmits the digital signal, which has been converted by the A/D conversion unit 113, to the information display and feedback module 150 through each channel according to a DMA scheme. Alternatively, the DMA controller 114 may be implemented to transmit results analyzed by the biometric information analyzing/processing unit 115 (to be described) to the information display and feedback module 150. In this manner, the bio-electrical signal or the analysis results measured by the EMG signal processing module 110 are transmitted to the information display and feedback module 150 in real time through the DMA communication scheme, thus allowing the measured signal to be monitored in real time.

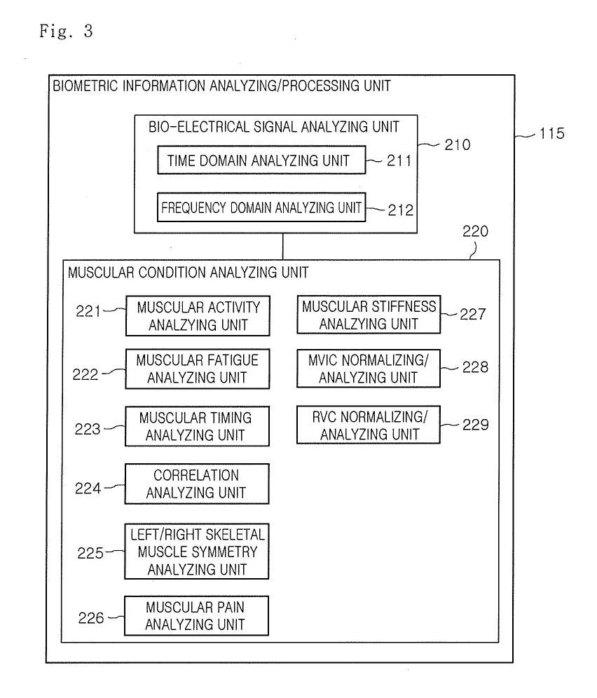

The biometric information analyzing/processing unit 115 analyzes the signal which has been detected by the EMG measurement unit 111, processed, and then analog-to-digital converted, to derive a meaningful value that can be used for correcting the spinal orientation. A detailed configuration and function of the biometric information analyzing/processing unit 115 will be described later with reference to FIG. 3.

The I/O interface 116 handles a data transmission and reception between the EMG signal processing module 110 and the HCl control module 130. Mainly, the I/O interface 116 serves to transmit the analysis results from the biometric information analyzing/processing unit 115 to the HCl control module 130.

The 3D angle detection module 120 detects angle information required for operating a spin correction device with respect to each individual patient spine and provides the detected angle information to the HCl control module 130. The 3D angle detection module 120 may be implemented as an MRI, CT, X-ray, or the like.

The HCl control module 130, which serves to generate a control signal for controlling the spinal orientation correction module 140 based on the information received from the EMG signal processing module 110 and the 3D angle detection module 120 and deliver the generated control signal to the spinal orientation correction module 140, includes an input unit 131, a setting unit 132, a controller 133, and an output unit 134.

The input unit 131 serves to receive information from the EMG signal processing module 110 and the 3D angle detection module 120.

The setting unit 132 serves to set execution conditions, perform functions, and the like, required for controlling the spinal orientation correction module 140, and in this case, the setting unit 132 may set the execution conditions, the performing function, or the like, according to information input by a user through the information display and feedback module 150.

The controller 133 generates a signal for controlling the spinal orientation correction module 140 according to the execution conditions or performing functions previously set by the setting unit 132 by using information received from the input unit 131.

The output unit 134 delivers the control signal generated by the controller 133 to the spinal orientation correction module 140.

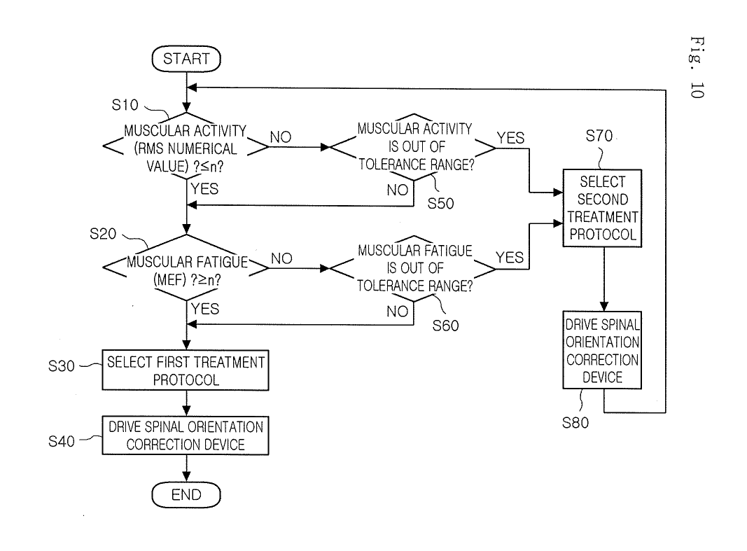

A detailed operational example of the HCl control module 130 will be described later with reference to FIG. 10.

The spinal orientation correction module 140, which serves to correct a patient's mal-aligned spine to its correct orientation, includes a spinal correction device controller 141 and a spinal correction device driving unit 142.

The spinal correction device controller 141 controls the spinal correction device driving unit 412 according to the control signal delivered from the HCl control module 130 to perform a spin correction treatment.

The spinal correction device driving unit 142 actually drives the spinal correction device under the control of the spinal correction device controller 141 to perform the spinal correction treatment on the patient. The spinal correction device driving unit 412 may be implemented by include a cervical area driving unit, an upper chest driving unit, a lower chest driving unit, a lumbar area driving unit, a pelvis area driving unit, a lower limb driving unit, and the like, the spin correction device driving unit 142 may be controlled such that two driving units operate to become separated from each other, a portion of a driving unit is lifted or lowered, a driving unit is entirely lifted or lowered, a driving unit is horizontally twisted, a driving unit simultaneously performs two or more of the above operations, or two or more driving units simultaneously perform different operations organically. Also, the spinal correction device driving unit 142 may be implemented by using an existing device for spinal correction.

The information display and feedback module 150 receives and displays information detected or analyzed by the EMG signal processing module 110 in real time so that the healer can monitor information regarding the patient's muscular condition while performing spinal correction treatment. Also, the information display and feedback module 150 receives information regarding the execution conditions, performing function, or the like, required for controlling the spine orientation correction module 140 from the user and delivers the received information to the HCl control module 130.

FIG. 3 is a detailed block diagram of the biometric information analyzing/processing unit illustrated in FIG. 2. The biometric information analyzing/processing unit 115 includes a bio-electrical signal analyzing unit 210 and a muscular condition analyzing unit 220.

The bio-electrical signal analyzing unit 210, which serves to analyze a received non-processed EMG signal through a standardized analysis scheme to derive various numeric values used for analyzing the muscular condition, includes a time domain analyzing unit 211 and a frequency domain analyzing unit 212.

The time domain analyzing unit 211 analyzes the received non-processed EMG signal in a time domain. In detail, the time domain analyzing unit 211 derives values of an integral average, an RMS (Root Mean Square), a PTP (Post-Tetanic Potential), an MEF (Median Edge Frequency), and an MDF (medial frequency).

Here, the Integral Average(VI.A) and RMS(VRMS) indicate an amplitude information (uV) of an EMG signal waveform and muscular activity. The integral average is obtained by taking absolute values of N number of non-processed EMG signals and averaging them as shown in Equation 1. The RMS is obtained by squaring the N number of non-processed EMG signals, averaging them, and extracting the square root of the averaged value. In this case, N indicates the number of the non-processed EMG signals, and v(t) indicates an EMG signal value at time t.

$$\begin{matrix}{V}_{I . A .} = \frac{1}{N} {\sum }_{i = 1}^{N} \vert v ( {t}_{i} ) \vert & [ Equation 1 ] \\ {V}_{RMS} = \sqrt{\frac{1}{N} {\sum }_{i = 1}^{N} {v}^{2} ( {t}_{i} )} & [ Equation 2 ]\end{matrix}$$

PTP(VPTP) indicates the amplitude of M-wave and, as shown in Equation 3, it refers to the difference between a maximum value and a minimum value among M-wave values measured until before a next stimulus is applied after stimulus is applied once. In this case, Vmax refers to the maximum value of M-wave, and Vmin refers to the minimum value of M-wave.

V_{PTP}=┐Vₘₐₓ−Vₘᵢₙ┐ [Equation 3]

MEF(fMEF) refers to the frequency of a portion at which an integral value of a power spectrum of an EMG signal is precisely halved, when the power spectrum is obtained. Namely, when the power spectrum is integrated by using the MEF as a reference as shown in Equation 4 below, values of both sides are equal. The MEF is calculated by using data until before a next stimulus is applied after stimulus is applied once, and S(f) refers to the density of the power spectrum of v(t).

∫₀^{f}^{_{MEF}}S(f)df=∫f_{_{MEF}}^{∞}S(f)df [Equation 4]

MDF(fMDF) refers to a frequency calculated by dividing the total amount, which is integrated after multiplying a frequency value itself to the power spectrum value of each frequency when the power spectrum of the EMG signal is obtained, by the integral value of the power spectrum. The MDF(fMDF) is represented by Equation 5 shown below. The MDF is calculated by using data until before a next stimulus is applied after stimulus is applied once, and S(f) refers to the density of the power spectrum of v(t).

$${f}_{MDF} = \frac{{\int }_{0}^{∞} f \cdot S ( f ) d f}{{\int }_{0}^{∞} S ( f ) d f} \qquad [ Equation 5 ]$$

The frequency domain analyzing unit 212 analyzes the received non-processed EMG signal in a frequency domain, namely, analyzes the characteristics of the power spectrum. In detail, the frequency domain analyzing unit 212 performs an SEF (Spectral Edge Frequency) analysis, an MEF (Median Edge Frequency) analysis, a statistical analysis, a correlation analysis, and filtering.

The SEF analysis refers to a corresponding particular frequency value (unit: Hz) when the area from a low edge (the left in a frequency axis) to a particular frequency value accounts for a ‘a certain % of the area of the entire frequency domain’ in the power spectrum, such as SEF-95%, SEF-90%, SEF-50%(MEF), SEF-25%, and the like.

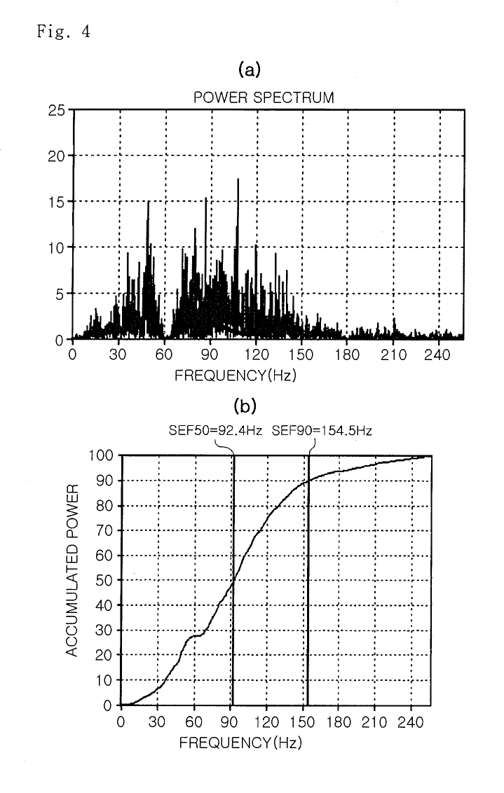

FIG. 4 is a graph for explaining the SEF analysis performed by the frequency domain analyzing unit illustrated in FIG. 3. After a normalized accumulated power distribution is obtained between 0 to 100% as shown in FIG. 4(b) from the power spectrum illustrated in FIG. 4(a), a frequency value of x-axis where a corresponding accumulated power value at the y axis corresponding to a certain % can be checked and obtained.

Also, muscular fatigue can be recognized through the MEF analysis. The MEF is in inverse proportion to muscular fatigue. Namely, when muscular fatigue increases, the MEF is lowered. This is because, when muscles are fatigued, an electrical refractory period of muscular cells is lengthened, so as the muscular fatigue is increased, high frequency is reduced and low frequency component becomes dominant. Also, because the MEF value is equal to SEF-50%, the muscular fatigue can be recognized through the SEF analysis.

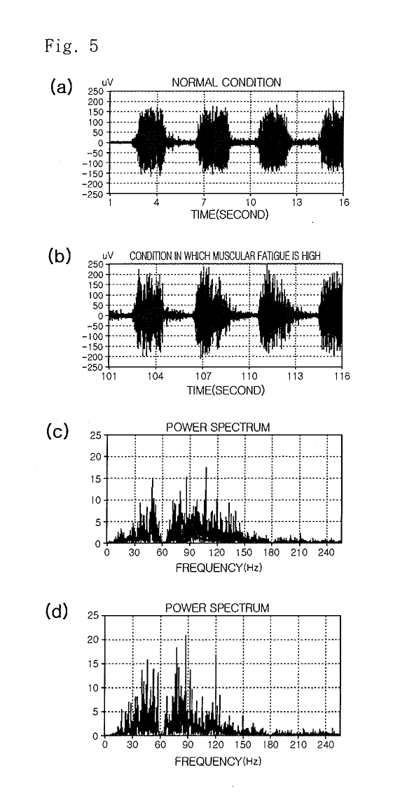

FIG. 5 is a graph for explaining an MEF analysis performed by the frequency domain analyzing unit illustrated in FIG. 3. FIG. 5(a) illustrates a non-processed EMG signal when muscles in a normal condition are contracted, FIG. 5(b) illustrates a non-processed EMG signal when a muscular fatigue is high, FIG. 5(c) illustrates a power spectrum when muscles in a normal condition are contracted, and FIG. 5(d) illustrates the power spectrum when muscles with a high level of fatigue are contracted.

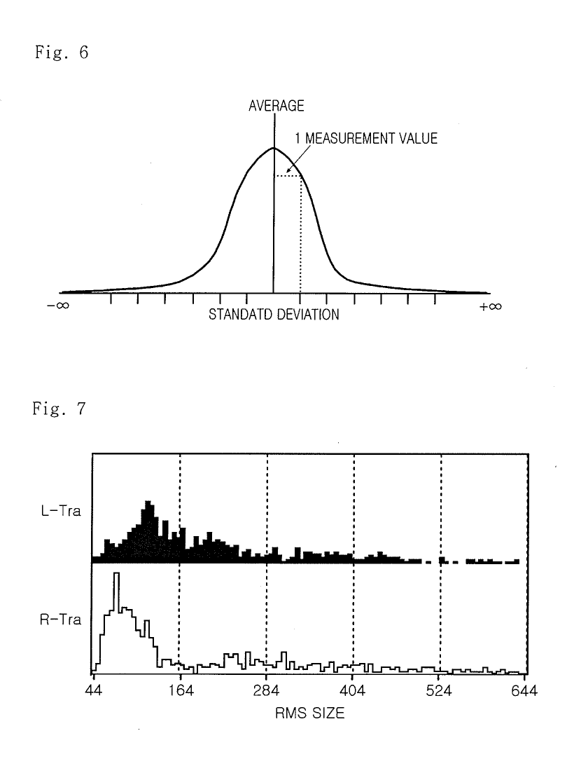

Statistical analysis can be performed by obtaining mean, standard deviation, skewedness, and kurtosis from the probability distribution graph illustrated in FIG. 6.

The mean (i.e., average) is an average value of measurement values on the probability distribution graph as shown in Equation 6 below, including offset information.

$$\overline{x} = \frac{1}{N} {\sum }_{j = 1}^{N} {x}_{j} ( {x}_{1} , {x}_{2} , … , {x}_{N} ) \qquad [ Equation 6 ]$$

The standard deviation refers to the degree of spreading of the probability distribution, including amplitude information. The standard deviation is obtained according to Equation 7 and Equation 8 shown below:

$$\begin{matrix}σ = {S}_{d} ( {x}_{1} , {x}_{2} , … , {x}_{N} ) = \sqrt{Var ( {x}_{1} , {x}_{2} , … , {x}_{N} )} & [ Equation 7 ] \\ Var ( {x}_{1} , {x}_{2} , … , {x}_{N} ) = \frac{1}{N - 1} {\sum }_{j = 1}^{N} {( {x}_{j} - \overline{x} )}^{2} & [ Equation 8 ]\end{matrix}$$

Skewedness refers to the degree of asymmetry (+: rightward direction, −: leftward direction) of the probability distribution, which is obtained according to Equation 9 shown below:

$$Skewness = \frac{1}{N} {\sum }_{j = 1}^{N} {[ \frac{{x}_{j} - \overline{x}}{σ} ]}^{3} \qquad [ Equation 9 ]$$

The kurtosis refers to the degree of sharpness of the probability distribution, which is obtained according to Equation 10 shown below:

$$Kurtosis = \frac{1}{N} {\sum }_{j = 1}^{N} {[ \frac{{x}_{j} - \overline{x}}{σ} ]}^{4} - 3 \qquad [ Equation 10 ]$$

A correlation analysis indicates the relevance between two types of interrelated data. A covariance of two types of interrelated data having N number of data equally as shown in Equation 11 below, which is then divided by the deviation of each interrelated data, to obtain the relevance.

$$\begin{matrix}p = \frac{Cov ( x , y )}{{σ}_{x} \cdot {σ}_{y}} \\ = \frac{\frac{1}{N - 1} {\sum }_{i = 1}^{N} ( {x}_{i} - {μ}_{x} ) \cdot ( {y}_{i} - {μ}_{x} )}{\sqrt{\frac{1}{N - 1} {\sum }_{j = 1}^{N} {( {x}_{j} - {μ}_{x} )}^{2}} \cdot \sqrt{\frac{1}{N - 1} {\sum }_{j = 1}^{N} {( {y}_{j} - {μ}_{x} )}^{2}}} \\ = \frac{{\sum }_{i = 1}^{N} ( {x}_{i} - {μ}_{x} ) \cdot ( {y}_{i} - {μ}_{x} )}{\sqrt{{\sum }_{j = 1}^{N} {( {x}_{j} - {μ}_{x} )}^{2}} \cdot \sqrt{{\sum }_{j = 1}^{N} {( {y}_{j} - {μ}_{x} )}^{2}}}\end{matrix} \qquad [ Equation 11 ]$$

Filtering is performed to filter out only a waveform of a particular band, when only the waveform of the particular band is desired to be observed or when an analysis method is desired to be applied only to the waveform of the particular band, in performing preprocessing to cancel noise caused by a surrounding environment. The filter may be implemented by utilizing a mathematical algorithm such as FFT, IIR, FIR, and the like, and may be implemented as a low pass filter (LPF), a high pass filter (HPF), a band pass filter (BPF), a band stop filter (BSF), a notch filter, and the like.

The muscular condition analyzing unit 220, which serves to analyze the condition of muscles by using various numeric values derived by the bio-electrical signal analyzing unit 210, includes a muscular activity analyzing unit 221, a muscular fatigue analyzing unit 222, a muscular timing analyzing unit 223, a correlation analyzing unit 224, a left/right skeletal muscle symmetry analyzing unit, a muscular pain analyzing unit 226, a muscular stiffness analyzing unit 227, an MVIC (Maximum Voluntary Isomeric Contraction) normalizing/analyzing unit 228, and an RVC (Reference Voluntary Contraction) normalizing/analyzing unit 229.

The muscular activity analyzing unit 221 indicates muscular activity with the integral average and RMS value derived by the time domain analyzing unit 211.

The muscle fatigue analyzing unit 222 indicates muscular fatigue with a value derived by an integrated EMG (IEMG) and median frequency (MEF) analysis method.

In this case, the IEMG reflects the number of motor unit recruitments, indicators of the amount of activity (or performance) of muscles and a change in a firing frequency by full-wave-rectifying an EMG signal. The number of motor unit recruitments and firing frequency are calculated by Equation 12 shown below. In this case, E(t) refers to the EMG, and T refers to a contraction duration.

$$I E M G = \frac{1}{T} {\int }_{0}^{T} \vert E ( t ) \vert d t \qquad [ Equation 12 ]$$

For reference, in order to recognize how much each of the muscles around the spine contributes to overall muscular strength, the level of contribution of each of the muscles around the spine may be calculated by normalizing the integrated EMG as represented by Equation 13 shown below. IN this case, Mx refers to muscles of x=1,3,5 (spine part).

$$Level of contribution of Mx = ( Mx / {\sum }_{i = 1 , 3 , 5} Mi ) \times 100 \qquad [ Equation 13 ]$$

The median frequency (MEF) is calculated by Equation 14 shown below to quantify the muscular fatigue, in which S(f) refers to the density of power spectrum.

$${\int }_{0}^{{f}_{MEF}} S ( f ) d f = {\int }_{{f}_{MEF}}^{∞} S ( f ) d f = \frac{1}{2} {\int }_{0}^{∞} S ( f ) d f \qquad [ Equation 14 ]$$

The reduction rate of the MEF refers to the fatigue of muscles, so the MEF is quantified according to Equation 15 shown below. In this case, i=1, 3, 5 (spine part), and j=1, 2, . . . , 13, and IMED indicates an initial MEF.

DR(Lᵢ)=100−Min{MEF(Lᵢ, Rᵢ)}/IMF(Lᵢ)×100 [Equation 15]

The muscular timing analyzing unit 223 calculates a point in time when muscles start to be contracted. The muscular timing analyzing unit 223 calculates the point in time by using an RMS value and a standard deviation value of each interval.

The correlation analyzing unit 224 quantifies the relevance between EMG signals measured from different portions. The correlation analyzing unit 224 may be able to recognize how much degree the contraction patterns of two muscles are similar at a muscular contraction timing of two muscular portions, when a corresponding operation performed. Also, The correlation analyzing unit 224 calculates it by the Pearson algorithm as shown in Equation 16 below:

$$r = \frac{{\sum }_{i} ( {x}_{i} - \overline{x} ) ( {y}_{i} - \overline{y} )}{\sqrt{{\sum }_{i} {( {x}_{i} - \overline{x} )}^{2}} \sqrt{{\sum }_{i} {( {y}_{i} - \overline{y} )}^{2}}} \qquad [ Equation 16 ]$$

The left/right skeletal muscle symmetry analyzing unit 225 analyzes the balance (i.e., symmetry) of left and right spinal muscles. The balance, which is the yardstick for determining the degree of a bad posture, is obtained by comparing the RMS values of erector spine muscles and deep muscle of respective spinal levels, which are symmetrical left and right.

The muscular pain analyzing unit 226 analyzes the sensitivity of muscles of the spine part to infer the degree of pain. The muscular pain analyzing unit 226 compares a change in frequency with the lapse of time to infer and analyze the degree of pain. For example, treatment frequency signals (e.g., the degree of amplitude, an aspect of the signals, etc.) before and after a medical treatment or those of a former day and next day may be compared to infer and analyze the degree of pain.

The muscular stiffness analyzing unit 227 analyzes the stiffness condition of muscles through a probability distribution. The stiffness degree of muscles are indicated by a probability distribution graph (the distribution, tilt, the degree of spreading, or the like, in the graph).

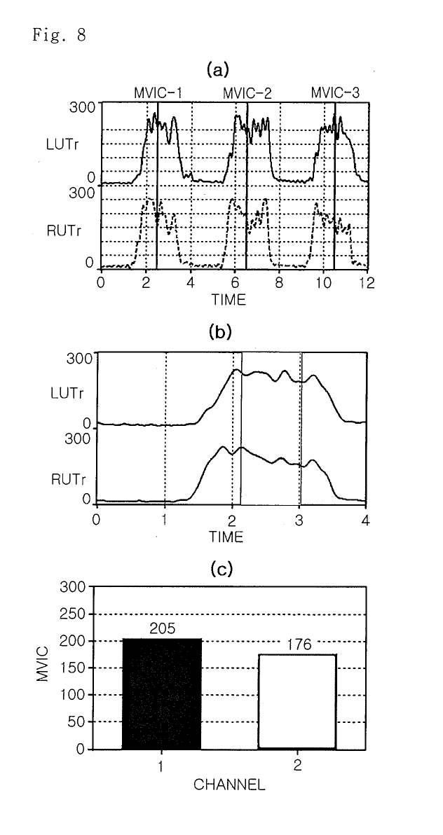

FIG. 7 is a graph for explaining an analysis process performed by a muscular stiffness analyzing unit illustrated in FIG. 3. For example, when a person who feels pain at his left shoulder performs typing for a certain duration, as shown in FIG. 7, the probability distribution of an EMG of muscles of the left shoulder is rightwardly lopsided, as compared with that of the muscles of the right shoulder.

The MVIC normalizing/analyzing unit 228 normalizes an MVIC value by excluding an unnecessary influence thereon to its maximum level. The MVIC normalizing/analyzing unit 228 determines a certain reference value and indicates how percentage level a measurement value corresponds to the reference value. A detailed calculation method is as follows.

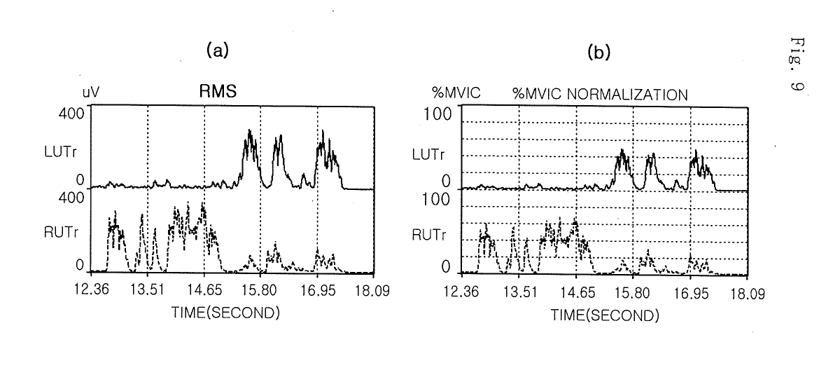

FIGS. 8 and 9 are graphs for explaining an analysis process performed by the MVIC normalizing/analyzing unit illustrated in FIG. 3. As shown in FIG. 8, an RMS value obtained when a subject (or a testee) applies a maximum isometric contraction to corresponding muscle is determined as a reference value, and in order to ensure reliability, for example, measurements are repeatedly performed three times to obtain an average to determine an MVIC. And then, as shown in FIG. 9, an RMS value in a checking operation is divided by the MVIC, which is then multiplied by 100 to calculate % MVIC unit.

$$\% M V I C = \frac{RMS}{M V I C} \times 100 \qquad [ [ Equation 17 ]$$

The RVC normalizing/analyzing unit 229 performs the same operation as that of the MVIC normalizing/analyzing unit 228, except that the MVIC normalizing/analyzing unit 228 determines an RMS value obtained when a contraction determined based on a desired reference is applied to corresponding muscle, as a reference value.

FIG. 10 is a flow chart illustrating a treatment process by the system for correcting a spinal orientation through an analysis of bio-electrical signals of muscles according to an exemplary embodiment of the present invention. Specifically, FIG. 10 shows the process of performing a medical treatment protocol fitting the patient's condition by controlling the spinal orientation correction module 140 by the HCl control module 130 by using the information regarding the muscular activity and the muscular fatigue derived by the EMG signal processing module 110 as described above.

When the muscular activity value (or the RMS value) is smaller than a pre-set certain value (step S10) and the muscular fatigue value (or the MEF value) is higher than a pre-set certain value (step S20), a first treatment protocol including treatment operations fitting such a situation is selected (step S30) and the spinal correction device is driven according (step S40).

Meanwhile, when any one of the muscular activity and the muscular fatigue does not satisfy the pre-set conditions and is out of a tolerance range (steps S50 and S60), a second treatment protocol configured to be suitable for the situation is selected (step S70) and the spinal correction device is driven accordingly (step S80). In this case, the spinal correction is repeatedly performed according to the second treatment protocol until such time as both the muscular activity and the muscular fatigue satisfy the tolerance range, and when both the muscular activity and the muscular fatigue satisfy the tolerance range, the spinal correction according to the first treatment protocol is performed.

While the present invention has been shown and described in connection with the exemplary embodiments, it will be apparent to those skilled in the art that modifications and variations can be made without departing from the spirit and scope of the invention as defined by the appended claims.

What is claimed is:

1. A system for correcting spinal orientation through an analysis of bio-electrical signals of muscles, the system comprising:
an electromyography (EMG) signal processing module measuring bio-electrical signals of muscles related to a spinal disease and analyzing the same;
an HCl control module generating a control signal for controlling a spinal orientation correction module based on information received from the EMG signal processing module and delivering the generated control signal to the spinal orientation correction module;
the spinal orientation correction module correcting a mal-aligned spine of a patient to its correct orientation according to the control signal; and
an information display and feedback module receiving information from the EMG signal processing module in real time, displaying the received information, and delivering information received from a user to the HCl control module.

2. The system of claim 1, further comprising:
a three-dimensional (3D) angle detection module detecting information regarding a spine angle of the patient required for controlling the spinal orientation correction module and providing the detected information to the HCl control module.

3. The system of claim 1, wherein the EMG signal processing module comprises:
an EMG measurement unit detecting muscular bio-electrical signals through a surface recording electrode or a static EMG scanner ceramic;
an EMG receiving/processing unit preprocessing a signal received from the EMG measurement unit;
an analog-to-digital (A/D) conversion unit converting an analog signal, which has been preprocessed by the EMG receiving/processing unit, into a digital signal;
a biometric information analyzing/processing unit analyzing the converted digital signal to calculate clinical numerical value indicating a muscular condition of a patient;
a DMA (Digital Media Adapter) controller transmitting the digital signal which has been converted by the A/D conversion unit or the analysis results obtained by the biometrical information analyzing/processing unit to the information display and feedback module according to a DMA method; and
an I/O interface for transmitting and receiving data between the EMG signal processing module and the HCl control module.

4. The system of claim 1, wherein the HCl control module comprises:
an input unit receiving information from the EMG signal processing module;
a setting unit setting execution conditions or performing function information required for controlling the spinal orientation correction module;
a controller generating a control signal for controlling the spinal orientation correction module according to the execution conditions or performing function information previously set by the setting unit by using information received from the input unit; and
an output unit delivering the control signal generated by the controller to the spinal orientation correction module.

5. The system of claim 1, wherein the spinal orientation correction module comprises:
a spinal correction device controller controlling a spinal correction device driving unit according to a control signal received from the HCl control module; and
the spinal correction device driving unit driving a spinal correction device to perform a spinal correction treatment on the patient.

6. The system of claim 3, wherein the biometric information analyzing/processing unit comprises:
a bio-electrical signal analyzing unit analyzing an EMG signal which has been converted into the digital signal to derive clinical numerical values for analyzing a muscular condition of the patient; and
a muscular condition analyzing unit analyzing a muscular condition of the patient by using the clinical numerical values derived by the bio-electrical signal analyzing unit.

7. The system of claim 6, wherein the biometrical information signal analyzing/processing unit comprises:
a time domain analyzing unit analyzing the EMG signal in a time domain to derive values of an integral average, an RMS (Root Mean Square), a PTP (Post-Tetanic Potential), an MEF (Median Edge Frequency), and an MDF (Medial Frequency); and
a frequency domain analyzing unit analyzing the EMG signal in a frequency domain to perform an SEF (Spectral Edge Frequency) analysis, an MEF (Median Edge Frequency) analysis, a statistical analysis, a correlation analysis, and filtering.

8. The system of claim 7, wherein the muscular condition analyzing unit comprises:
a muscular activity analyzing unit indicating a muscular activity by the integral average and RMS values derived by the time domain analyzing unit;
a muscular fatigue indicating muscular fatigue by a value derived according to an IEMT and an MEF (Median Frequency) analysis method;
a muscular timing analyzing unit calculating a point in time when a muscular contraction starts to take place;
a correlation analyzing unit quantifying relevance between EMG signals measured at different portions;
a left/right skeletal muscle symmetry analyzing unit analyzing the balance of left and right spinal muscles;
a muscular pain analyzing unit analyzing sensitivity of spine part muscle to infer and analyze the degree of pain;
a muscular stiffness analyzing unit analyzing a stiff condition of muscle through a probability distribution of an EMG signal;
an MVIC normalizing/analyzing unit normalizing and calculating an MVIC value; and
an RVC normalizing/analyzing unit normalizing and calculating an RVC value.

9. The system of claim 1, wherein when the muscular activity and the muscular fatigue values satisfy pre-set conditions, the HCl control module drives the spinal correction device according to a first treatment protocol, and when one of the muscular activity and the muscular fatigue values does not satisfy the pre-set conditions and is out of a tolerance range, the HCl control module provides control to repeatedly drive the spinal correction device according to a second treatment protocol until such time as both the muscular activity and the muscular fatigue values satisfy the tolerance range.

10. A method for correcting a spinal orientation, the method comprising:
measuring and analyzing bio-electrical signals of muscles related to a spine disease;
generating a control signal for controlling a spinal orientation correction device according to the results obtained by analyzing the bio-electrical signal; and
driving the spinal orientation correction device according to the control signal to correct a mal-aligned spine of a patient to its orientation.

11. The method of claim 10, further comprising:
detecting information regarding a spine angle of the patient,
wherein, in the generating of the control signal, the control signal is generated additionally with reference to the detected information regarding the spine angle.

12. The method of claim 10, wherein the measuring and analyzing of the bio-electrical signal comprises:
detecting bio-electrical signals of muscles;
preprocessing the detected bio-electrical signal;
converting the preprocessed signal into a digital signal; and
analyzing the converted digital signal to calculate a clinical numerical value indicating a muscular condition of the patient.

13. The method of claim 12, wherein the calculating the clinical numerical value comprises:
analyzing the digital signal in a time domain to derive values of an integral average, an RMS (Root Mean Square), a PTP (Post-Tetanic Potential), an MEF (Median Edge Frequency), and an MDF (Medial Frequency); and
analyzing the digital signal in a frequency domain to perform an SEF(Spectral Edge Frequency) analysis, an MEF (Median Edge Frequency) analysis, a statistical analysis, a correlation analysis, and filtering.

14. The method of claim 13, wherein the calculating of the clinical numerical value comprises:
calculating a muscular activity by the integral average and RMS values;
calculating a muscular fatigue by a value derived according to an IEMT and an MEF (Median Frequency) analysis method;
calculating a point in time when a muscular contraction starts to take place;
quantifying relevance between EMG signals measured at different portions;
analyzing the balance of left and right spinal muscles;
analyzing sensitivity of spine part muscle to infer and analyze the degree of pain;
analyzing a stiff condition of muscle through a probability distribution of an EMG signal;
normalizing and calculating an MVIC value; and
normalizing and calculating an RVC value.

15. The method of claim 10, wherein when the muscular activity and the muscular fatigue values satisfy pre-set conditions, a spinal correction device is driven according to a first treatment protocol, and when one of the muscular activity and the muscular fatigue values does not satisfy the pre-set conditions and is out of a tolerance range, the spinal correction device is controlled to be repeatedly driven according to a second treatment protocol until such time as both the muscular activity and the muscular fatigue values satisfy the tolerance range.
